(19) United States
(12) Patent Application Publication
(10) Pub. No.: US 2006/0293602 A1
(43) Pub. Date: Dec. 28, 2006

(54) Sleep management device

(76) Inventor: Richard Charles Clark, Kalamunda (AU)

Correspondence Address: INTELLECTUAL PROPERTY LAW GROUP LLP, 12 SOUTH FIRST STREET, SUITE 1205, SAN JOSE, CA, 95113, US

(21) Appl. No.: 10/553,286

(22) PCT Filed: Apr. 8, 2004
(86) PCT No.: PCT/AU04/00009

§ 371 Date: Oct. 14, 2005

(30) Foreign Application Priority Data
Apr. 16, 2003 (AU) 2003901877

(51) Int. Cl. A61B 5/02; A61B 5/00

(52) U.S. Cl. 600/500; 600/300

(57) ABSTRACT

A short sleep/nap management apparatus and method are disclosed. The apparatus has sensor means (35) to detect one or more physiological parameters associated with a transition in sleep stages (13-19) from wakefulness (13), processing means (37, 39, 33) to process the parameters to determine when the transition is reached and start the timer to run for a predetermined period, and alarm means (47) to actuate at the end of said predetermined period to awaken the user.

### FIELD OF THE INVENTION

The present invention relates to a method and apparatus for management of sleep/naps.

### BACKGROUND ART

Research into sleep patterns, has determined that sleep, often considered as a time when the brain and body are simply turned off, is actually a complex physiological process and not a single homogeneous state. Normal sleep has been found to consist of two clearly different states that have different physiological mechanisms and indicators. These are commonly known as Rapid Eye Movement (REM) sleep and Non-Rapid Eye Movement (NREM) sleep.

The NREM sleep state is sometimes referred to as quiet sleep (QS). The NREM sleep state is characterized by slowed physiological and mental activity. Heart rate, breathing, and brain activity slow, and no dreaming occurs. NREM sleep has been classified into four stages, with stage 1 being the shallowest and stage 4 being the deepest sleep. Referring to FIG. 1, a graph illustrates a representative sleep cycle. It should be emphasised that no two individuals are the same, and that the relative times for each stage as shown in FIG. 1 will vary from individual to individual. The REM sleep state is shown at 11, while the more lightly shaded stages are NREM sleep state.

Commencing from wakefulness 13, in the process of falling asleep, a subject will progress in a cycle comprising some four stages of NREM sleep (note that other analyses identify a larger number of stages characterised by more subtle physiological changes, but the four stage model discussed here is generally well recognised internationally), interrupted by REM sleep. The four stages of the NREM sleep state comprise Stage 1 indicated at 15 which can be equated to deep drowsiness, Stage 2 indicated at 17 which can be equated to light sleep, Stage 3 indicated at 19 which can be equated to deep sleep, and Stage 4 indicated at 21 which can be equated to very deep sleep.

Both slow wave sleep (SWS) and ‘delta sleep’ are terms often used to refer to Stages 3 and 4 only.

REM sleep, 11, by contrast, is often called active sleep. It is marked by accelerated respiration, increased brain activity, rapid eye movement and muscle relaxation. During REM sleep, the sleeper is physiologically and mentally active (dreaming), while physically paralysed.

Periods of NREM and REM sleep typically alternate throughout each sleep period in an 80-120 minute cycle, with roughly ⅔rds NREM sleep followed by ⅓rd REM sleep. A normal sleep cycle consists of the sequence: waking (13), NREM stages 1 (15), 2 (17), 3 (19), 4 (21), 3 (19), 2 (17), REM (11).

Sleep debt is an increasing problem in the modern world. Having to tailor sleeping hours to the external demands of local and international commuting, family commitments, office hours, project timelines, international phone calls, crises and so on is debilitating and destructive to ones health. A natural aid to managing sleep debt is the short day sleep, sometimes called a power nap. It can be very refreshing and invigorating when it works out well. Unfortunately it is often difficult to manage your nap/sleep in order to awake refreshed. If you set your alarm clock for 15 minutes and then don't get to sleep for 12 minutes the sleep will be frustratingly short and mostly ineffective. If you set the alarm clock for 90 minutes and fall asleep straight away then you may be woken up from deep Stage 3 or Stage 4 sleep and feel groggy and confused due to sleep inertia. If the sleep is too long then it may make getting to sleep at night difficult and perpetuate the sleep debt problem.

Every individual and every circumstance is different in terms of how long it will take to get to sleep. Every individual has their own nap/sleep pattern in terms of how quickly they progress from starting to fall asleep until they enter deep sleep. Furthermore, there is some variability between individuals in the physiologically observable indicators that manifest during various sleep stages.

Various devices have been described in patent specifications for systems that monitor physiologically observable signs that manifest during various sleep stages, for example through monitoring EEG traces, Heart Rate (inter beat) Variability, skin galvanic response, muscle tonus and twitching, eyelid blinking, electrical potential, temperature changes and so forth.

These systems invariably are attempting to determine sleep state automatically. Distinguishing between various sleep states or stages by external monitoring of internal conditions is a subtle process. The changes in signals are often difficult to identify and require careful calibration for each individual by an external operator. This is exacerbated by variability between individuals in physiologically observable signs that manifest during various sleep stages.

These devices are invariably inconvenient (ie Head mounted electrodes); complex, difficult to set up, sensitive and require training and independent monitoring to ensure the device is correctly attached and calibrated. Thus, the devices hitherto known or described that attempt to definitively identify a sleep stage, and then act on the presumed identified stage are going to experience a degree of unreliability, unless attachment and operation is supervised by an experienced technician.

Some devices have been described, which are intended to be operated by the user, rather than a technician. For example, U.S. Pat. No. 5,928,133 describes an apparatus and method for awakening a user during a preset time interval or bracket at the point when, for all intents and purposes, the user is already awake. The described apparatus monitors the user to determine when the user is close to wakefulness, as is the case slightly before or immediately after REM sleep, and then wakes the user when this sleep stage has been detected.

U.S. Pat. No. 4,228,806 discloses a sleep state inhibited wake-up alarm. This alarm has a settable wake-up time and will inhibit issuance of an alarm signal if the user is in a deep sleep or in a REM sleep state, up to a point in time when the alarm will issue. Thus the alarm of U.S. Pat. No. 4,228,806 provides some flexibility to the normal wake up time of a typical alarm clock, so as not to awaken the user if the user is in a deep sleep or REM sleep.

Similarly U.S. Pat. No. 5,101,831 monitors pulse rate to ensure that a user awakens after REM sleep state.

These cases appear to demonstrate that the easiest sleep state to reliably detect is the REM sleep state. However, the detection of the REM sleep state is of little use for a nap/short sleep management device, as the time to reach this state is, for the most part, beyond the desired time for a nap/short sleep. None of the devices described above is capable of operation as a simple alarm to awaken a user, and thereby manage short sleep times, for example during work hours, or at other times of the day. In fact, it appears to be common to all devices described in prior-publications, the user passes through at least one deep sleep stage, and REM stage sleep or light sleep after REM stage sleep is detected before the user is awakened. Most of the devices are also designed to awaken the user from a prolonged sleep of from 6 to 10 hours (i.e. in the morning).

It is an object of this invention to provide a short sleep/nap management device, which is user operable, and with some practice, easily adaptable to different users.

It is a preferred object of the present invention to provide a short sleep/nap management device that is inexpensive, dependable and fully effective in accomplishing its intended purpose.

Throughout the specification, unless the context requires otherwise, the word “comprise” or variations such as “comprises” or “comprising”, will be understood to imply the inclusion of a stated integer or group of integers but not the exclusion of any other integer or group of integers.

### DISCLOSURE OF THE INVENTION

In accordance with one aspect of the present invention there is provided a short sleep/nap management apparatus comprising sensor means to detect one or more physiological parameters associated with a transition in sleep stages from wakefulness, processing means to process said parameters to determine when said transition is reached and start a timer to run for a predetermined period, and alarm means to actuate at the end of said predetermined period. When the alarm means actuates, the person using the sleep management apparatus will be awakened.

Preferably said transition is any point in time from the onset of stage 1 or stage 2 sleep, to an event preceding onset of stage 3 sleep.

Thus, also in accordance with the present invention there is provided a short sleep/nap management apparatus comprising sensor means to detect one or more physiological parameters associated with a transition in sleep stages from wakefulness, said transition being any point in time from the onset of stage 1 or stage 2 sleep, to an event preceding onset of stage 3 sleep, processing means to process said parameters to determine when said transition is reached and start a timer to run for a predetermined period, and alarm means to actuate at the end of said predetermined period. When the alarm means actuates, the person using the sleep management apparatus will be awakened.

Preferably said predetermined period is user adjustable. The predetermined period is the time that the user desires to sleep once the transition is reached. This time will be adjusted by trial and error, with the object being to time awakening to avoid going into deep sleep (NREM stages 3 and 4), and thus avoid long duration sleep inertia. Some users may find that they do not suffer sleep inertia if they reach stage 3, however they would want to avoid the very deep sleep of stage 4.

Preferably said sleep management apparatus includes a second timer to run for a second predetermined period, wherein said alarm means actuates at the end of said second predetermined period. The second predetermined period is a maximum time, preferably user selectable, that the user desires to allocate for a sleep, regardless of whether a sleep is achieved. This is analogous to a time set by a normal alarm clock, and avoids too much time being taken if the time to reach the transition takes longer than initially expected.

Preferably said transition is any point in time from the onset of stage 1 sleep, to an event preceding onset of stage 2 sleep.

Preferably said transition is a point in time at or shortly after the onset of stage 1 sleep.

Preferably said transition point is user adjustable. In this manner, the sleep management apparatus of the invention provides, in its most preferred form, the ability for the user to select the most appropriate sleep “trigger” event for them.

A wide variety of physiological data could be used to accomplish the purpose of the invention, such as ECGs, EEGs, movement sensors, galvanic skin response, or any other of the common parameters monitored by sleep researchers, or electrical potential change or temperature change.

Preferably said sensor means senses one or more of heart/pulse rate, and respiration rate.

Preferably said sensor means senses heart/pulse rate.

Preferably said one or more parameters detected is a significant change in average heart rate (SCAHR). By “significant”, “sustained” is meant, rather than “transient” or “temporary”.

Also in accordance with the present invention there is provided a method of achieving a short sleep or nap comprising detecting one or more physiological parameters associated with a transition in sleep stages from wakefulness, determining when said transition is reached and timing a predetermined period, and actuating alarm means at the end of said predetermined period. When the alarm means actuates, the person using the sleep management apparatus will be awakened.

Preferably said transition is any point in time from the onset of stage 1 or stage 2 sleep, to an event preceding onset of stage 3 sleep.

Thus, also in accordance with the present invention there is provided a method of achieving a short sleep or nap comprising detecting one or more physiological parameters associated with a transition in sleep stages from wakefulness, said transition being any point in time from the onset of stage 1 or stage 2 sleep, to an event preceding onset of stage 3 sleep, determining when said transition is reached and timing a predetermined period, and actuating alarm means at the end of said predetermined period. When the alarm means actuates, the person using the sleep management apparatus will be awakened.

Preferably said method provides for said predetermined period to be user adjustable. The predetermined period is the time that the user desires to sleep once the transition is reached. This time will be adjusted by trial and error, with the object being to time awakening to avoid going into deep sleep (NREM stages 3 and 4), and thus avoid long duration sleep inertia. Some users may find that they do not suffer sleep inertia if they reach stage 3, however they would want to avoid the very deep sleep of stage 4.

Preferably said method includes providing a second timer to run for a second predetermined period, wherein said alarm means actuates at the end of said second predetermined period. The second predetermined period is a maximum time, preferably user selectable, that the user desires to allocate for a sleep, regardless of whether a sleep is achieved. This is analogous to a time set by a normal alarm clock, and avoids too much time being taken if the time to reach the transition takes longer than initially expected.

Preferably said transition is any point in time from the onset of stage 1 sleep, to an event preceding onset of stage 2 sleep.

Preferably said transition is a point in time at or shortly after the onset of stage 1 sleep.

Preferably said method provides for said transition point to be user adjustable. In this manner, the sleep management apparatus of the invention provides, in its most preferred form, the ability for the user to select the most appropriate sleep “trigger” event for them.

A wide variety of physiological data could be used to accomplish the purpose of the invention, such as ECGs, EEGs, movement sensors, galvanic skin response, or any other of the common parameters monitored by sleep researchers, or electrical potential change or temperature change.

Preferably said detecting of said transition utilises sensor means to senses one or more of heart/pulse rate, and respiration rate.

Preferably said sensor means senses heart/pulse rate.

In its most preferred form, the present invention provides a novel wake-up alarm in which the user is provided with a monitor that automatically identifies SCAHR events and allows the setting of a user selected delay time and subsequent alarm that will trigger while they are asleep.

Through use of the apparatus the user learns which of the SCAHR trigger events are easiest to detect and are consistently repeatable in their individual case. They can then tailor the subsequent post event sleep duration to best suit their personal sleep patterns and current circumstances, ie time available, recent sleep history, anticipated sleep deficit due to a long future wakefulness requirement etc. Continued use of the apparatus and examination of settings and effects allows the user to refine their understanding of their sleep patterns and of the appropriate settings for their most reliable triggers and their current circumstances.

Preferably said apparatus includes monitoring means to record said one or more parameters, as a function with time. Preferably said apparatus can produce a chart from said monitoring means. Thus, the user can view their average heart rate variation charts on previous occasions and determine which of the SCAHR events are most consistent in their patterns. The user can then select one or more of those events as the trigger for the start of the countdown to the alarm.

Similarly the user can use previous average heart rate charts and records of matching subsequent alertness/performance to determine the most beneficial time delay from the selected SCAHR event(s).

With the ability to select the transition point or trigger event and the predetermined period, the user can tailor the duration of their actual asleep time to provide the most benefit from the sleep. This can only be determined when the trigger event has occurred. The time at which this trigger event will occur could not be known when preparing for sleep, only once it has occurred.

By monitoring the physiological changes of the user during the sleep cycle the device can identify a trigger event and curtail the duration of subsequent sleep the user is allowed. The time selected by the user/operator is set to avoid progressing to a deep sleep stage and the consequent significant sleep inertia that would entail.

### BRIEF DESCRIPTION OF THE DRAWINGS

A preferred embodiment of the invention will now be described with reference to the drawings, in which:

FIG. 2 is a view of part of the embodiment fitted to a user;

FIG. 3 is a functional block diagram of the apparatus of the embodiment;

FIG. 4 is a graphical representation of the heart rate as it changes over time during sleep, identifying some of the key change SCAHR events; and

FIG. 5 is a block diagram showing the logical operation of the embodiment.

### BEST MODE(S) FOR CARRYING OUT THE INVENTION

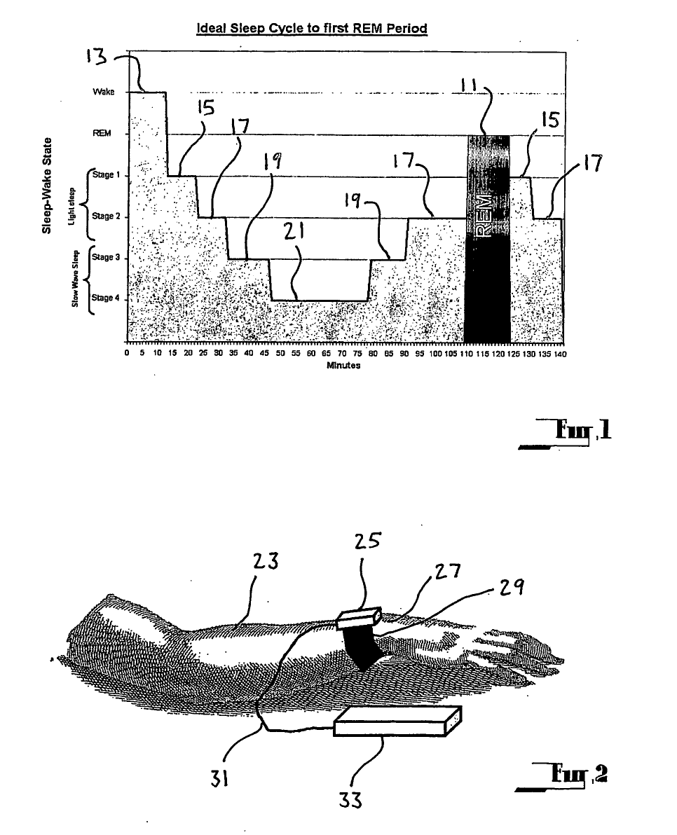

The embodiment is a sleep management device that allows the user to set the maximum time from detection of a sleep event, that the user is allowed to sleep. Referring to FIG. 2 the user's arm 23 has a transducer 25 attached at the wrist 27 by a band 29, a connecting wire cable 31 and the control and processing unit 33.

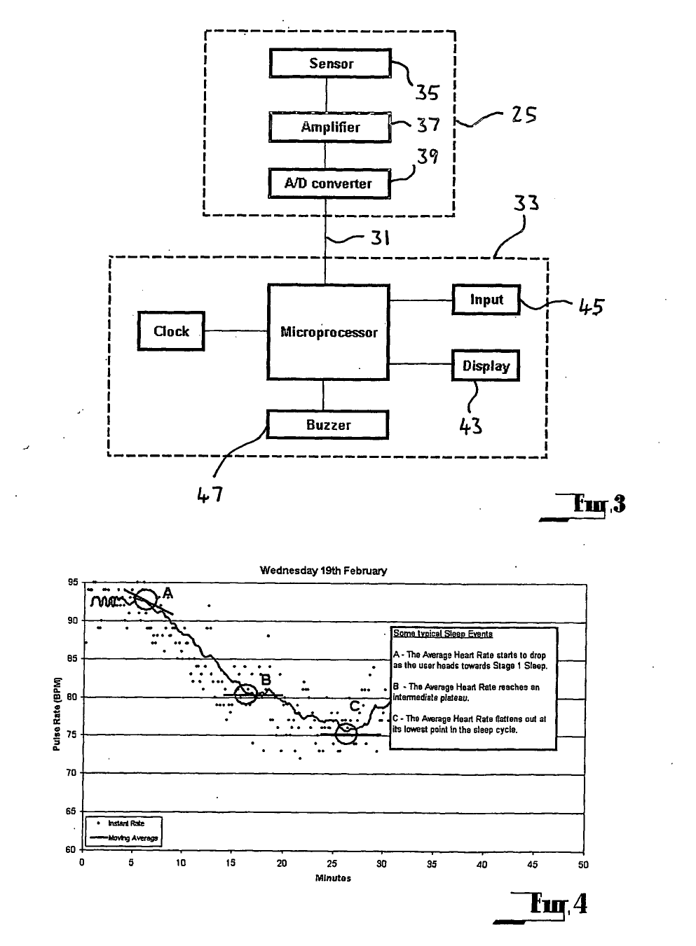

Referring to FIG. 3 the transducer 25 consists of a sensor 35, an amplifier 37 and an analogue to digital converter 39. The sensor 35 is an infrared photoelectric sensor which detects changes in tissue blood volume. A suitable sensor is a model no. MLT1020 IR Plethysmograph produced by AD Instruments Pty Ltd.

As an alternative, a sensor can be fabricated using a high power low angle spread infra-red LED and a phototransistor or a photodiode, both mounted on a base which is secured to a wrist-strap. The LED can be a Lumex device, part no OED-EL-8L, which is a 3 mm device with a transparent lens. The photodiode can be a side viewing device manufactured by Bright LED and sold under their part no BPD-RQ0ADV1. The LED and photodiode are available from Dick Smith Electronics in Australia, the photodiode being sold under part no Z1956. The LED and photodiode are spaced apart by between 1 cm and 3 cm, and aimed with beam/view-path intersecting at between 1 cm and 2 cm from the devices.

The sensor 35 has an infrared LED, which directs its output into the tissue, and an infrared detector that receives the infrared light after it has bounced back from the underlying bone within the tissue. The oxyhaemoglobin in the blood absorbs the infrared light in proportion to its volume. As the arterial pulse increases the blood flow through the tissue, the amount of infrared light received at the infrared detector is reduced. The infrared detector provides a small analogue electrical signal, which varies in proportion to the arterial pulse, to the amplifier 37. The amplifier 37 then increases this voltage to levels that can be detected and converted by an analogue to digital converter 39.

The amplifier 37 includes filtering to smooth the AC pulse signal into a sinusoidal waveform for greater reliability of conversion into meaningful data by the analogue to digital converter 39. The analogue to digital converter 39 converts the amplified and filtered/smoothed pulse signal to digital values that can be manipulated and analysed by a microprocessor 41. The microprocessor can derive and show on the display 43, information such as pulse rate, and pulse rate as a function with time. The information relating to pulse rate is stored in a memory associated with the microprocessor 41. The information relating to pulse rate can be called up and reviewed by the user.

An input device comprising a keypad 45 allows the user to call up data and input a desired transition point at which a timer should start, and input the predetermined time that the timer should run before the user should be awakened. The processing device 33 includes a buzzer 47 to awaken the user at the end of the predetermined period.

The user can also input data relating to a second predetermined period, corresponding to the absolute latest time that the buzzer 47 should sound, in the event that the transition occurs too late and the user would otherwise sleep beyond a required absolute time. (ie a known appointment time).

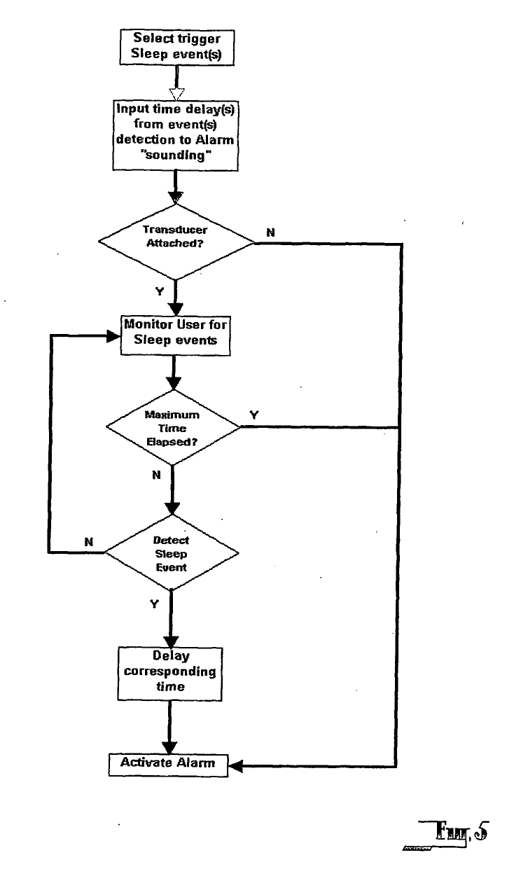

The keypad 45 allows the user to call up data relating to pulse rate, such as the graph shown in FIG. 4. From this graph, the user can deduce that at point A, the average heart rate begins to drop as the user begins to fall asleep. By trial and error, the user can see and select the appropriate SCAHR event(s) and the predetermined periods/time delay(s) beyond these events at which the buzzer 47 should sound. The object is for the user to select a predetermined period which is not so long that the user falls into a deep or REM sleep, thus minimising sleep intertia.

Further analyses of the changes of the average heart beat rate by the microprocessor 41 allows it to determine when SCAHR events have occurred. Determination of SCAHR events is as follows. The microprocessor 41 receives digital values from the analogue to digital converter 39 that samples the amplified pulse waveform signal every 25 milliseconds. The microprocessor 41 determines the approximate peaks of this signal by identifying when the slope of the line between two sequential sample values drops below zero. These pulse peak values are compared to the moving average pulse peak values both for relative magnitude and for inter-peak times. If the peak values or inter-peak times are more than +/−50% different from the moving averages then the peak is an artefact of movement by the user due to rapid movement of the arm or significant re-orientation of the body. These artefact peak values are invalid.

The peak values are collected across an approximately 10 second epoch. If all the peaks within the epoch are valid then the time across this set of peaks, from first peak to last peak within the epoch, is divided by the number of peaks minus 1 to give an average interbeat time in seconds. Dividing 60 seconds per minute by this value gives the average heart rate in beats per minute. The changing values of average heart rate, and the times at which they occur, are accumulated in an array. A linear regression analysis is performed on a number of sequential average heartbeat values and corresponding times from this array. This analysis yields the slope of the line of best fit for these points. These slopes are monitored on a moving basis. By determining when these slopes have changed significantly it is possible to identify SCAHR events. For example when the slope changes from close to horizontal to clearly downwards (−0.25 bpm/minute or steeper) then the average heart rate has started to drop. The user is then close to sleep onset. This can be seen as point A in FIG. 4. This point is in the middle of the slope line. Similarly some individuals have a clear plateau in the drop in heartrate when the slope of the line nears the horizontal (shallower than −0.05 bpm/minute) as in Point B in FIG. 4. This point is in the middle of slope line. This plateau is an identifiable SCAHR for this individual.

The slope of the line after the plateau is again downwards (−0.25 bpm/minute or steeper) until the heartrate reaches its lowest point in the sleep cycle. At this point the slope of the heartrate again becomes close to horizontal (shallower than −0.05 bpm/minute) and after a period of time the slope will start upwards (positive slope greater than 0 bpm/minute). These changes in slope identify the lowest moving average heart rate as shown at C in FIG. 4. This point is in the middle of the slope line that is close to horizontal. The time at which these SCAHR events occur can then be used selectively by the user as alarm trigger points in their sleep cycle.

When the SCAHR event that matches the user selected SCAHR event has occurred, the microprocessor 41 counts down the user specified predetermined period with reference to the internal clock associated with the microprocessor 41 and then gives the alarm signal using the buzzer 47.

While one particular analysis has been described in relation to the most preferred embodiment, it will be understood that other methods of analysing the changes of the average heart beat may be utilised in alternative embodiments.

It will be understood that all of the functions of the processing unit 33 may be performed by a PDA style hand-held computer, having the appropriate program. A flowchart for such a program is shown in FIG. 5.

The embodiment of the invention allows the user to have a short sleep or nap, and achieves this by limiting the amount of time the user is asleep, through timing a short sleep/nap period from the point in time the user passes through significant indicators in pulse rate change, from wakefulness as the user falls asleep. In effect, the timer runs from a point where the user has just fallen asleep or is nearly asleep, and is set by trial and error by the user, with the aim that the user avoids a deep sleep stage, and is awoken in a refreshed state. The second timer ensures that the user does not sleep beyond a predetermined absolute point in time in the event that onset of sleep is delayed, thereby ensuring that the user does not miss an appointment through having had difficulty falling asleep.

It should be appreciated that the invention is not limited to the particular embodiment disclosed herein, and that changes may be made without departing from the spirit and scope of the invention. For example, in alternative embodiments, the alarm signal can be any of a number of outputs which may include, without limitation, audible alarm sounds, flashing lights, relaying a signal to another device etc.

In an alternative embodiment, the cable 31 (connecting the A/D converter 39 to the microprocessor 41) could be replaced by wireless transceivers (either radio or infrared) so that user movement is unrestricted. In such an arrangement, to minimise power consumption, a microprocessor could be included in the transducer 25, so that part of the changes in average heat beat analysis are computed in the transducer, and only rate of change information (and preferably only significant rate of change information) is transmitted from the transducer 25 to the control and processing unit 33, rather than continual transmission of heart beat data. The microprocessor 41 in the control and processing unit 33 would then only perform the decision making aspects of the system.

In such arrangements utilising wireless transceivers, the transducer 25 may also incorporate rechargeable batteries, and the control and processing unit 33 may incorporate either a connection to allow charging of the rechargeable batteries, or a docking station so that the rechargeable batteries can be charged by power inducted by inductive coupling between the transducer 25 and the control and processing unit 33. Furthermore, in yet a further embodiment, the entire assembly can be minimised such that it all fits in a watch-like enclosure that fits on to the wrist.

It is possible to monitor and use sleep events other than SCAHR. For example there are a wide variety of physiological characteristics such as ECGs, EEGs, movement sensors, galvanic skin response, Heart Rate (inter beat) Variability, muscle tonus and twitching, eyelid blinking, electrical potential, temperature changes or any other of the common parameters monitored by sleep researchers that could be utilised to determine and detect the event. It would also be possible to use multiple events and multiple matching delays so that the resultant delay is the shortest of the combinations of events detected and subsequent delays assigned.

1. A short sleep/nap management apparatus comprising sensor means to detect one or more physiological parameters associated with a transition in sleep stages from wakefulness, processing means to process said parameters to determine when said transition is reached and start a timer to run for a predetermined period, and alarm means to actuate at the end of said predetermined period.

2. A short sleep/nap management apparatus comprising sensor means to detect one or more physiological parameters associated with a transition in sleep stages from wakefulness, said transition being any point in time from the onset of stage 1 or stage 2 sleep, to an event preceding onset of stage 3 sleep, processing means to process said parameters to determine when said transition is reached and start a timer to run for a predetermined period, and alarm means to actuate at the end of said predetermined period.

3. A short sleep/nap management apparatus as claimed in claim 1 wherein said predetermined period is user adjustable.

4. A short sleep/nap management apparatus as claimed in claim 1 wherein said sleep management apparatus includes a second timer to run for a second predetermined period to an absolute time, wherein said alarm means actuates at the end of said second predetermined period.

5. (canceled)

6. (canceled)

7. (canceled)

8. A short sleep/nap management apparatus as claimed in claim 1 wherein said sensor means senses heart/pulse rate.

9. (canceled)

10. (canceled)

11. (canceled)

12. A method of achieving a short sleep or nap comprising detecting one or more physiological parameters associated with a transition in sleep stages from wakefulness, determining when said transition is reached and timing a predetermined period, and actuating alarm means at the end of said predetermined period.

13. A method as claimed in claim 12, wherein said transition is any point in time from the onset of stage 1 or stage 2 sleep, to an event preceding onset of stage 3 sleep.

14. A method as claimed in claim 12 including providing for said predetermined period to be user adjustable.

15. A method as claimed in claim 12 including providing a second timer to run for a second predetermined period, wherein said alarm means actuates at the end of said second predetermined period.

16. A method as claimed in claim 12 wherein said transition is any point in time from the onset of stage 1 sleep, to an event preceding onset of stage 2 sleep.

17. A method as claimed in claim 12 wherein said transition is a point in time at or shortly after the onset of stage 1 sleep.

18. A method as claimed in claim 12 including providing for said transition point to be user adjustable.

19. A method as claimed in claim 12 wherein the detecting of said transition utilises sensor means to sense heart/pulse rate.

20. (canceled)

21. A short sleep/nap management apparatus as claimed in claim 2 wherein said predetermined period is user adjustable.

22. A short sleep/nap management apparatus as claimed in claim 2 wherein said sleep management apparatus includes a second timer to run for a second predetermined period to an absolute time, wherein said alarm means actuates at the end of said second predetermined period.

23. A short sleep/nap management apparatus as claimed in claim 2 wherein said transition is any point in time from the onset of stage 1 sleep, to an event preceding onset of stage 2 sleep.

24. A short sleep/nap management apparatus as claimed in claim 2 wherein said transition is a point in time at or shortly after the onset of stage 1 sleep.

25. A short sleep/nap management apparatus as claimed in claim 2 wherein said transition point is user adjustable.

26. A short sleep/nap management apparatus as claimed in claim 2 wherein said sensor means senses heart/pulse rate.

27. A short sleep/nap management apparatus as claimed in claim 26 wherein said one or more parameters detected is a significant change in average heart rate, being sustained rather than transient or temporary.
